(19) United States
(12) Patent Application Publication
(10) Pub. No.: US 2024/0325761 A1
(43) Pub. Date: Oct. 3, 2024

(54) NEUROSTIMULATION WITH PHASE OFFSET BETWEEN MODULATED PARAMETERS

(71) Applicant: Boston Scientific Neuromodulation Corporation, Valencia, CA (US)

(72) Inventors: Brandon Thio, Durham, NC (US); Tianhe Zhang, Studio City, CA (US); Jessica Block, Los Angeles, CA (US)

(21) Appl. No.: 18/621,815

(22) Filed: Mar. 29, 2024

Related U.S. Application Data
(60) Provisional application No. 63/455,460, filed on Mar. 29, 2023.

(51) Int. Cl. A61N 1/36; A61N 1/05; A61N 1/372

(52) U.S. Cl. CPC A61N 1/36196; A61N 1/05; A61N 1/36142; A61N 1/36157; A61N 1/36171; A61N 1/36175; A61N 1/37247

(57) ABSTRACT

A system may include a neuromodulator and a programmer configured to program the neuromodulator to deliver neuromodulation according to a modulated neurostimulation parameter setting. The modulated neurostimulation parameter setting may include a first modulated neurostimulation parameter waveform and a second modulated neurostimulation parameter waveform, and a phase offset between the first and the second modulated neurostimulation parameter waveforms.

### CLAIM OF PRIORITY

This application claims the benefit of U.S. Provisional Application No. 63/455,460 filed on Mar. 29, 2023, which is hereby incorporated by reference in its entirety.

### TECHNICAL FIELD

This document relates generally to medical systems, and more particularly, but not by way of limitation, to systems, devices, and methods for providing neurostimulation according to a modulated neurostimulation parameter setting that includes a phase offset between modulated neurostimulation parameter waveforms.

### BACKGROUND

Medical devices may include therapy-delivery devices configured to deliver a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such as pacemakers to treat cardiac arrhythmia, defibrillators to treat cardiac fibrillation, heart failure cardiac resynchronization therapy devices, cochlear stimulators to treat deafness, retinal stimulators to treat blindness, muscle stimulators to produce coordinated limb movement, spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc. A neurostimulation device (e.g., DBS, SCS, PNS or TENS) may be configured to treat pain. By way of example and not limitation, a DBS system may be configured to treat tremor, bradykinesia, and dyskinesia and other motor disorders associated with Parkinson's Disease (PD).

Neurostimulation energy may be delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Many current neurostimulation systems are programmed to deliver periodic pulses with one or a few uniform waveforms continuously or in bursts. However, the human nervous systems use neural signals having much more sophisticated patterns to communicate various types of information, including sensations of pain, pressure, temperature, etc. The nervous system may interpret an artificial stimulation with a simple pattern of stimuli as an unnatural phenomenon, and respond with an unintended and undesirable sensation and/or movement. For example, some neurostimulation therapies are known to cause paresthesia and/or vibration of non-targeted tissue or organ.

Nonregular temporal (e.g., non-tonic) patterns of neuromodulation can improve the efficacy of therapy or make side effects more tolerable. For example, a dynamic stimulation pattern has been proposed where a stimulation parameter (amplitude, pulse width, or rate) is modulated by a sinusoidal envelope. Such dynamic stimulation patterns may be preferred compared to tonic stimulation.

Improved systems for providing effective therapy using non-tonic neurostimulation are desired.

### SUMMARY

Various embodiments of the present subject matter provide a phase offset between at least two simultaneous modulation of multiple stimulation parameters. The phase offset between the modulation of these parameters may be important in determining effective stimulation protocols. Some embodiments may modulate more than two stimulation parameters, and may provide phase offsets between each unique pair of the modulated stimulation parameters. The phase offset between simultaneously modulating multiple stimulation parameters may be used to determine optimal dynamic stimulation parameters for controlling complex parameters of stimulation beyond what is directly programmable such as current injection and the amount of population activation.

An example (e.g., “Example 1”) of a system may a neuromodulator and a programmer configured to program the neuromodulator to deliver neuromodulation according to a modulated neurostimulation parameter setting. The modulated neurostimulation parameter setting may include a first modulated neurostimulation parameter waveform and a second modulated neurostimulation parameter waveform, and a phase offset between the first and the second modulated neurostimulation parameter waveforms.

In Example 2, the subject matter of Example 1 may optionally be configured such that the programmer includes a user interface configured to receive a user input corresponding to the phase offset. The programmer may include a controller operably connected to the user interface to determine the phase offset based on the user input.

10010) In Example 3, the subject matter of Example 2 may optionally be configured such that the user input includes an objective for the neurostimulation parameter setting, the method further comprising determining the phase offset from the objective.

In Example 4, the subject matter of any one or more of Examples 1-3 may optionally be configured such that the programmer is configured to access a neurostimulation setting having at least a first neurostimulation parameter and a second neurostimulation parameter, modulate the first parameter to provide the first modulated neurostimulation parameter waveform, and modulate the second parameter to provide the second modulated neurostimulation parameter waveform.

In Example 5, the subject matter of Example 4 may optionally be configured such that both the first neurostimulation parameter and the second neurostimulation parameter are modulated using a sinusoidal function.

In Example 6, the subject matter of Example 5 may optionally be configured such that the programmer includes a user interface configured to receive user input for at least one of a modulation frequency or a modulation depth. At least one of the first modulated neurostimulation parameter waveform and the second modulated neurostimulation parameter waveform may be at least partially defined using the received user input for the at least one of the modulation frequency or the modulation depth.

In Example 7, the subject matter of Example 6 may optionally be configured such that the programmer is configured to display in a single display screen on the user interface the first modulated neurostimulation parameter waveform, the second modulated neurostimulation parameter waveform, and the phase offset.

In Example 8, the subject matter of any one or more of Examples 4-7 may optionally be configured such that the programmer includes a user interface configured to receive user input used to program the neurostimulation setting, including a first value for the first neurostimulation parameter and a second value for the second neurostimulation parameter.

In Example 9, the subject matter of any one or more of Examples 4-8 may optionally be configured such that the first and the second neurostimulation parameters include two or more of an amplitude, a pulse width, a frequency, charge per pulse, a stimulation ON/OFF duty cycle, or electrode fractionalization.

In Example 10, the subject matter of any one or more of Examples 1-9 may optionally be configured such that the first and the second modulated neurostimulation parameter waveforms have a same modulation frequency.

In Example 11, the subject matter of any one or more of Examples 1-9 may optionally be configured such that the first and the second modulated neurostimulation parameter waveforms have a different modulation frequency.

In Example 12, the subject matter of Example 11 may optionally be configured such that the system is configured to enforce a minimum offset difference between the first and the second modulated neurostimulation parameter waveforms with the different modulation frequency.

In Example 13, the subject matter of any one or more of Examples 1-12 may optionally be configured such that the processor is configured to receive a user input to determine a waveform shape for a modulation function used to modulate at least one of the first neurostimulation parameter and the second neurostimulation parameter.

In Example 14, the subject matter of any one or more of Examples 1-13 may optionally be configured such that at least one of the programmer or the neurostimulator is configured to implement safety rules to prevent the modulated neurostimulation parameter setting from causing an unsafe dose of neurostimulation, and is configured to display a warning on a user interface if user input would cause the modulated neurostimulation parameter setting to be unsafe.

In Example 15, the subject matter of any one or more of Examples 1-14 may optionally be configured such that the neuromodulator includes an implantable spinal cord stimulator (SCS), an implantable deep brain stimulator (DBS), or a peripheral nerve stimulator (PNS).

Example 16 includes subject matter such as a method, means for performing acts, machine-readable medium including instructions that when performed by a machine cause the machine to perform acts, or an apparatus to perform. The subject matter may include accessing a first modulated neuromodulation parameter waveform and a second modulated neuromodulation parameter waveform, providing a phase offset between the first and the second modulated neurostimulation waveforms to provide a modulated neurostimulation setting that includes the first and the second modulated neurostimulation waveforms and the phase offset, and delivering neurostimulation according the modulated neurostimulation parameter setting.

In Example 17, the subject matter of Example 16 may optionally be configured to further include receiving a user input corresponding to the phase offset, and determining the phase offset based on the user input.

In Example 18, the subject matter of Example 17 may optionally be configured such that the user input includes an objective for the neurostimulation parameter setting, the method further comprising determining the phase offset from the objective.

In Example 19, the subject matter of any one or more of Examples 16-18 may optionally be configured to further include accessing a neurostimulation setting having at least a first neurostimulation parameter and a second neurostimulation parameter, and modulating the first parameter to provide the first modulated neurostimulation parameter waveform and modulating the second parameter to provide the second neurostimulation modulated parameter waveform.

In Example 20, the subject matter of Example 19 may optionally be configured such that both the first neurostimulation parameter and the second neurostimulation parameter are modulated using a sinusoidal function.

In Example 21, the subject matter of Example 20 may optionally be configured to further include receiving user input for at least one of a modulation frequency or a modulation depth. At least one of the first modulated neurostimulation parameter waveform and the second modulated neurostimulation parameter waveform may be at least partially defined using the received user input for the at least one of the modulation frequency or the modulation depth.

In Example 22, the subject matter of Example 21 may optionally be configured to further include displaying in a single display screen on a user interface the first modulated neurostimulation parameter waveform, the second modulated neurostimulation parameter waveform, and the phase offset.

In Example 23, the subject matter of any one or more of Examples 19-22 may optionally be configured to further include programming the neurostimulation setting. Programming the neurostimulation setting may include programming a first value for the first neurostimulation parameter and programming a second value for the second neurostimulation parameter.

In Example 24, the subject matter of any one or more of Examples 19-23 may optionally be configured such that the first and the second neurostimulation parameters include two or more of an amplitude, a pulse width, a frequency, charge per pulse, a stimulation ON/OFF duty cycle, or electrode fractionalization.

In Example 25, the subject matter of any one or more of Examples 16-23 may optionally be configured such that the first and the second modulated neurostimulation parameter waveforms have a same modulation frequency.

In Example 26, the subject matter of any one or more of Examples 19-25 may optionally be configured such that the first and the second modulated neurostimulation parameter waveforms have a different modulation frequency.

In Example 27, the subject matter of Example 26 may optionally be configured to further include enforcing a minimum offset difference between the first and the second modulated neurostimulation parameter waveforms with the different modulation frequency.

In Example 28, the subject matter of any one or more of Examples 16-27 may optionally be configured to further include receiving a user input to determine a waveform shape for a modulation function used to modulate at least one of the first neurostimulation parameter and the second neurostimulation parameter.

In Example 29, the subject matter of any one or more of Examples 16-28 may optionally be configured to further include delivering the neurostimulation according to safety rules to prevent the modulated neurostimulation parameter setting from causing an unsafe dose of neurostimulation, and displaying a warning on a user interface if user input would cause the modulated neurostimulation parameter to be unsafe.

Example 30 includes subject matter such as a machine-readable medium including instructions that when performed by a machine cause the machine to perform acts. The subject matter may include a non-transitory machine-readable medium including instructions, which when executed by a machine, cause the machine to perform a method that includes accessing a first modulated parameter waveform and a second modulated parameter waveform, providing a phase offset between the first and the second modulated neurostimulation waveforms to provide a modulated neurostimulation setting that includes the first and the second modulated neurostimulation waveforms and the phase offset, and delivering neurostimulation according the modulated neurostimulation parameter setting.

In Example 31, the subject matter of Example 30 may optionally be configured such that the method further includes comprising receiving a user input corresponding to the phase offset, and determining the phase offset based on the user input.

In Example 32, the subject matter of any one or more of Examples 30-31 may optionally be configured such that the method further includes accessing a neurostimulation setting having at least a first neurostimulation parameter and a second neurostimulation parameter, and modulating the first parameter to provide the first modulated neurostimulation parameter waveform and modulating the second parameter to provide the second neurostimulation modulated parameter waveform.

In Example 33, the subject matter of any one or more of Examples 30-32 may optionally be configured such that the first and the second modulated neurostimulation parameter waveforms have a same modulation frequency.

In Example 34, the subject matter of any one or more of Examples 30-33 may optionally be configured such that the first and the second modulated neurostimulation parameter waveforms have a different modulation frequency, and wherein the method further includes enforcing a minimum offset difference between the first and the second modulated neurostimulation parameter waveforms with the different modulation frequency.

In Example 35, the subject matter of any one or more of Examples 30-34 may optionally be configured such that the method further includes delivering the neurostimulation according to safety rules to prevent the modulated neurostimulation parameter setting from causing an unsafe dose of neurostimulation, and displaying a warning on a user interface if user input would cause the modulated neurostimulation parameter to be unsafe.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system.

FIG. 2 illustrates, by way of example, an example of an electrical therapy-delivery system.

FIG. 3 illustrates, by way of example and not limitation, the electrical therapy-delivery system of FIG. 2 implemented using an IMD.

FIG. 4 illustrates an example in which current and pulse width are modulated in phase with a same modulation function and further illustrates a corresponding injected charge for the waveform with the modulated current and modulated pulse width.

FIG. 5 illustrates an example in which current and pulse width are modulated out of phase with the same modulation function and further illustrates a corresponding injected charge for the waveform with the out-of-phase modulated current and modulate pulse width.

FIG. 6 illustrates an example in which modulated current and modulated pulse width are designed to provide a constant charge injection.

FIG. 7 illustrates, by way of example and not limitation, a programming screen display.

FIG. 8 illustrates, by way of example and not limitation, a modulated neurostimulation (or neuromodulation) setting.

FIG. 9 illustrates, by way of example and not limitation, preset parameter modulation settings designed to be effective in achieving neurostimulation objectives.

FIG. 10 illustrates, by way of example and not limitation, a screen display or a portion of screen display for programming or modifying parameter modulation settings.

FIG. 11 illustrates, by way of example and not limitation, a pulldown menu with parameters available to be selected for the first and/or second modulated parameters.

FIG. 12 illustrates, by way of example and not limitation, a slider bar that may be used to select the phase offset.

FIG. 13 illustrates, by way of example and not limitation, a screen display or a portion of screen display for programming or modifying parameter modulation settings.

FIG. 14 illustrates by way of example and not limitation, a screen display or a portion of screen display for programming or modifying parameter modulation settings.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

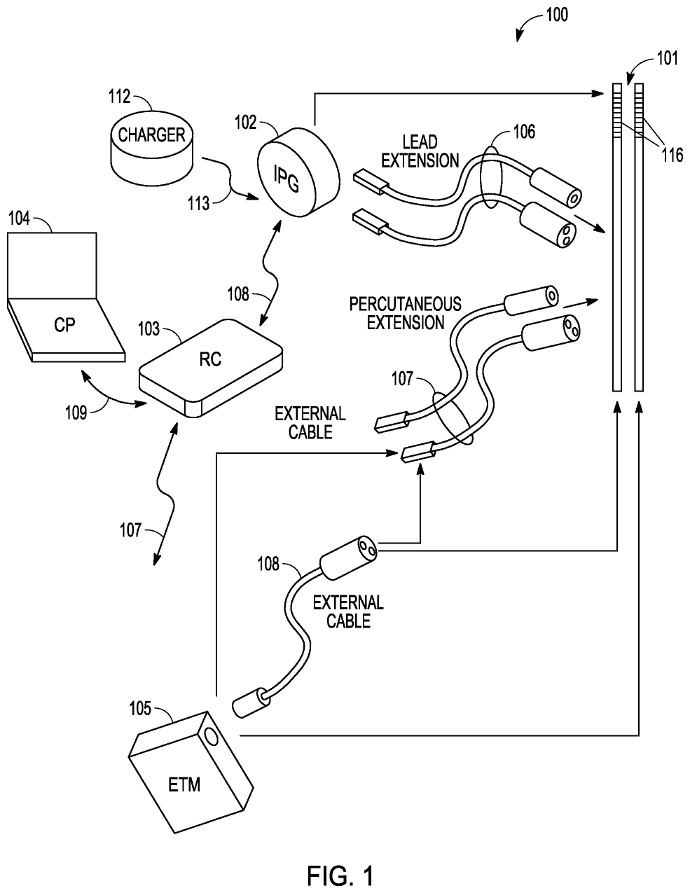

FIG. 1 illustrates, by way of example and not limitation, an electrical stimulation system 100. For example, the system may be used to deliver SCS, DBS or PNS. The electrical stimulation system 100 may be a neurostimulation system, and may generally include a one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. A surgical paddle lead can be used in place of one or more of the percutaneous leads. In some examples such as DBS systems, the leads 201 can be rotatable so that the electrodes 216 can be aligned with the target neurons after the neurons have been located such as based on the recorded signals. The electrodes 216 can include one or more ring electrodes, and/or one or more sets of segmented electrodes (or any other combination of electrodes). The IPG 102 includes pulse generation circuitry that delivers electrical modulation energy in the form of a pulsed electrical waveform (i.e., a temporal series of electrical pulses) to the electrodes in accordance with a set of modulation parameters. The waveform may include regular, irregular patterns of pulses, or other complex waveforms with shapes other than a rectilinear pulse.

The IPG may include an antenna allowing it to communicate bi-directionally with a number of external devices. The antenna may be a conductive coil within an IPG case or a header. When the antenna is configured as a coil, communication with external devices may occur using near-field magnetic induction. The IPG may also include a Radio-Frequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneous charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

A computing system (e.g., device(s)) may be used to create or modify waveforms, analyze sensor or user inputs, and/or used to program or control the operation of an electrical stimulation system. The computing system may include a processor, a memory, a display, and an input device. Optionally, the computing system may be separate from and communicatively coupled to neurostimulation system. Alternatively, the computing system may be integrated with the electrical stimulation system, such as part of the IPG 102, RC 103, CP 104, or ETM 105 illustrated in FIG. 1. The computing system can be a computer, tablet, mobile device, or any other suitable device for processing information. The computing system can be local to the user or can include components that are non-local to the computer including one or both of the processor or memory (or portions thereof). For example, the user may operate a terminal that is connected to a non-local processor or memory. The functions associated with the computing system may be distributed among two or more devices, such that there may be two or more memory devices performing memory functions, two or more processors performing processing functions, two or more displays performing display functions, and/or two or more input devices performing input functions. In some examples, the computing system can include a watch, wristband, smartphone, or the like. Such computing systems can wirelessly communicate with the other components of the neurostimulation system, such as the CP 104, RC 103, ETM 105, or IPG 102 illustrated in FIG. 1. The computing systems may be used for gathering patient information, such as general activity level or present queries or tests to the patient to identify or score pain, depression, stimulation effects or side effects, cognitive ability, or the like.

The processor may include one or more processors that may be local to the user or non-local to the user or other components of the computing device. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

In various examples, portions of the functions of the processor may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a processor that can receive and execute a set of instructions of performing the functions, methods, or techniques described herein.

The memory may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information, and which can be accessed by a computing device. Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display may be any suitable display or presentation device, such as a monitor, screen, display, or the like, and can include a printer. The display may be a part of a user interface configured to display information about stimulation settings (e.g., electrode configurations and stimulation parameter values and value ranges) and user control elements for programming a stimulation setting into an IPG.

The input device may be, for example, a keyboard, mouse, touch screen, track ball, joystick, voice recognition system, or any combination thereof, or the like. Another input device 430 may be a camera from which the clinician can observe the patient. Yet another input device 430 may a microphone where the patient or clinician can provide responses or queries.

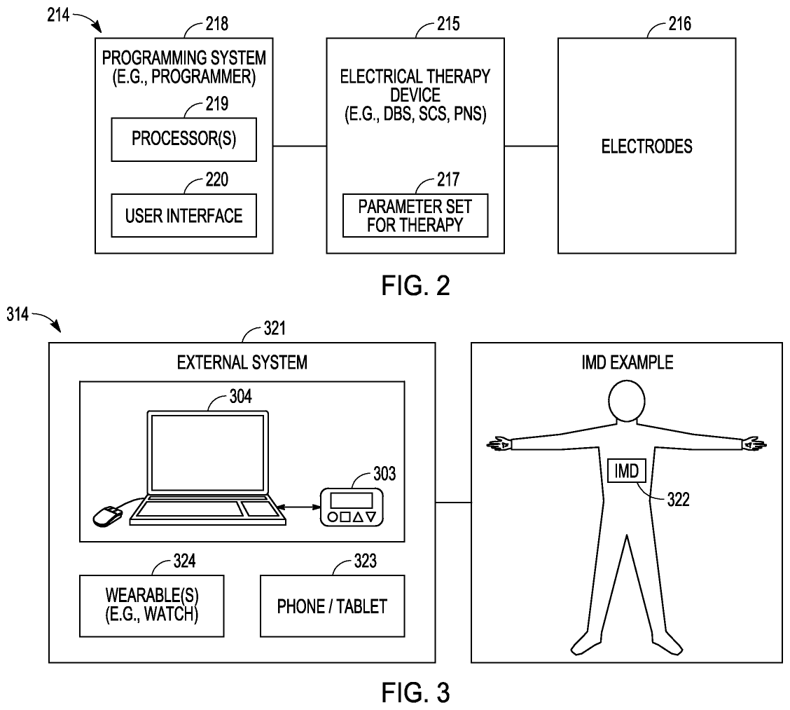

FIG. 2 illustrates, by way of example, an example of an electrical therapy-delivery system. The illustrated system 214 includes an electrical therapy device 215 configured to deliver an electrical therapy to electrodes 216 to treat a condition in accordance with a programmed parameter set 217 for the therapy. The system 214 may include a programming system 218, which may include at least part of a computing system. The programming system 218 may function as at least a portion of a processing system, that may include one or more processors 219 and a user interface 220. The programming system 218 may be used to program and/or evaluate the parameter set(s) used to deliver the therapy. The illustrated system 214 may be a DBS system, an SCS system or a PNS system.

The electrical therapy device 215 produces and delivers the neurostimulation. Neurostimulation pulses are provided herein as an example. However, the present subject matter is not limited to pulses, but may include other electrical waveforms (e.g., waveforms with different waveform shapes, waveforms with various pulse patterns). The electrical therapy device controls the delivery of the neurostimulation pulses using the plurality of neurostimulation parameters. One or more leads may each configured to be electrically connected to electrical therapy device 215 and a plurality of electrodes distributed in an electrode arrangement using the one or more leads. Each lead may have an electrode array consisting of two or more electrodes, which also may be referred to as contacts. Multiple leads may provide multiple electrode arrays to provide the electrode arrangement. The neurostimulation pulses are each delivered through a set of electrodes. The number of leads and the number of electrodes on each lead may depend on, for example, the distribution of target(s) of the neurostimulation and the need for controlling the distribution of electric field at each target.

The actual number and shape of leads and electrodes may vary for the intended application. An implantable waveform generator may include an outer case for housing the electronic and other components. An implantable pulse generator or IPG is a type of waveform generator. The outer case may be composed of an electrically conductive, biocompatible material, such as titanium, that forms a hermetically-sealed compartment wherein the internal electronics are protected from the body tissue and fluids. In some cases, the outer case may serve as an electrode (e.g., case electrode). The waveform generator may include electronic components, such as a controller/processor (e.g., a microcontroller), memory, a battery, telemetry circuitry, monitoring circuitry, neurostimulation output circuitry, and other suitable components known to those skilled in the art. The microcontroller executes a suitable program stored in memory, for directing and controlling the neurostimulation performed by the waveform generator. Electrical modulation energy is provided to the electrodes in accordance with a set of parameters programmed into the pulse generator. By way of example but not limitation, the electrical modulation energy may be in the form of a pulsed electrical waveform. Such parameters may comprise electrode combinations, which define the electrodes that are activated as anodes (positive), cathodes (negative), and turned off (zero), percentage of neurostimulation energy assigned to each electrode (fractionalized electrode configurations), and electrical pulse parameters, which define the pulse amplitude (measured in milliamps or volts depending on whether the pulse generator supplies constant current or constant voltage to the electrode array), pulse width (measured in microseconds), pulse rate (measured in pulses per second), and burst rate (measured as the modulation on duration X and modulation off duration Y). Electrodes that are selected to transmit or receive electrical energy are referred to herein as “activated,” while electrodes that are not selected to transmit or receive electrical energy are referred to herein as “non-activated.”

Electrical neurostimulation occurs between or among a plurality of activated electrodes, one of which may be the case of the waveform generator. The system may be capable of transmitting energy to the tissue in a monopolar or multipolar (e.g., bipolar, tripolar, etc.) fashion. Monopolar stimulation occurs when a selected one of the lead electrodes is activated along with the case of the waveform generator, so that energy is transmitted between the selected electrode and case. Any of the electrodes E1-E16 and the case electrode may be assigned to up to k possible groups or timing “channels.” In one embodiment, k may equal four. The timing channel identifies which electrodes are selected to synchronously source or sink current to create an electric field in the tissue to be stimulated. Amplitudes and polarities of electrodes on a channel may vary. In particular, the electrodes can be selected to be positive (anode, sourcing current), negative (cathode, sinking current), or off (no current) polarity in any of the k timing channels. The waveform generator may be operated in a mode to deliver electrical modulation energy that is therapeutically effective and causes the patient to perceive delivery of the energy (e.g., therapeutically effective to relieve pain with perceived paresthesia), and may be operated in a sub-perception mode to deliver electrical modulation energy that is therapeutically effective and does not cause the patient to perceive delivery of the energy (e.g., therapeutically effective to relieve pain without perceived paresthesia). The waveform generator may be configured to individually control the magnitude of electrical current flowing through each of the electrodes. For example, a current generator may be configured to selectively generate individual current-regulated amplitudes from independent current sources for each electrode. In some embodiments, the pulse generator may have voltage regulated outputs.

The system may be configured to stimulate or modulate spinal target tissue or other neural tissue. The configuration of electrodes used to deliver electrical pulses to the targeted tissue constitutes an electrode configuration, with the electrodes capable of being selectively programmed to act as anodes (positive), cathodes (negative), or left off (zero). In other words, an electrode configuration represents the polarity being positive, negative, or zero. An electrical waveform may be controlled or varied for delivery using electrode configuration(s). The electrical waveforms may be analog or digital signals. In some embodiments, the electrical waveform includes pulses. The pulses may be delivered in a regular, repeating pattern, or may be delivered using complex patterns of pulses that appear to be irregular. Other parameters that may be controlled or varied include the amplitude, pulse width, and rate (or frequency) of the electrical pulses. Each electrode configuration, along with the electrical pulse parameters, can be referred to as a “modulation parameter set.” Each set of modulation parameters, including fractionalized current distribution to the electrodes (as percentage cathodic current, percentage anodic current, or off), may be stored and combined into a modulation program that can then be used to modulate multiple regions within the patient.

The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. Furthermore, for example SCS systems may have thirty-two electrodes which exponentially increases the number of modulation parameters sets available for programming. To facilitate such selection, the clinician generally programs the modulation parameters sets through a computerized programming system to allow the optimum modulation parameters to be determined based on patient feedback or other means and to subsequently program the desired modulation parameter sets.

FIG. 3 illustrates, by way of example and not limitation, the electrical therapy-delivery system of FIG. 2 implemented using an implantable medical device (IMD). An IPG is a type of IMD. The illustrated system 314 includes an external system 321 that may include at least one programming device. The illustrated external system 321 may include a clinician programmer 304, similar to CP 104 in FIG. 1, configured for use by a clinician to communicate with and program the neuromodulator, and a remote control device 303, similar to RC 103 in FIG. 1, configured for use by the patient to communicate with and program the neuromodulator. For example, the remote control device 303 may allow the patient to turn a therapy on and off, change or select programs, and/or may allow the patient to adjust patient-programmable parameter(s) of the plurality of modulation parameters. FIG. 3 illustrates an IMD 322, although the therapy device may be an external device such as a wearable device. The external system 321 may include a network of computers, including computer(s) remotely located from the IMD 322 that are capable of communicating via one or more communication networks with the programmer 304 and/or the remote control device 303. The remotely located computer(s) and the IMD 322 may be configured to communicate with each other via another external device such as the programmer 304 or the remote control device 303. The remote control device 303 and/or the programmer 304 may allow a user (e.g., patient and/or clinician or rep) to answer questions as part of a data collection process. The external system 321 may include personal devices such as a phone or tablet 323, wearables such as a watch 324, sensors or therapy-applying devices. The watch may include sensor(s), such as sensor(s) for detecting activity, motion and/or posture. Other wearable sensor(s) may be configured for use to detect activity, motion and/or posture of the patient. The external system 321 may include, but is not limited to, a phone and/or a tablet. Notifications may be sent to the patient, physician, device rep or other users via the external system and through remote portals (e.g., web-based portals) provided by remote systems.

As provided above, a therapy may be delivered according to a parameter set that may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). These therapy parameters determine a stimulation pattern including temporal and spatial components of the pattern. The stimulation pattern may be a fixed or static pattern such as occurs when the therapy parameters are constant. The pattern may be a dynamic pattern such as occurs when at least one of therapy parameters is not constant (e.g., has a value that varies). For example, a dynamic pattern may be provided by implementing a modulating function (e.g., a sin wave or other function) on a therapy parameter.

Dynamic stimulation patterns may have two or more stimulation parameters modulated by a modulation function. For example, two or more of an amplitude, pulse width, pulse rate, charge per pulse, electrode fractionalization, or stimulation ON/OFF duty cycle may be modulated with a sin wave. When simultaneously modulating multiple parameters, the phase offset of the modulation waveforms may play a factor in determining the effects of stimulation.

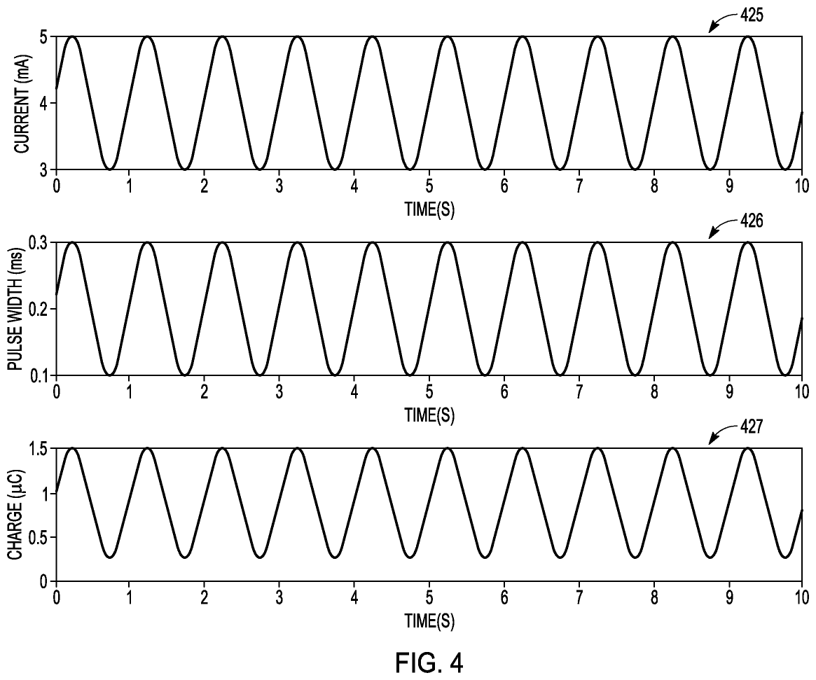

FIG. 4 illustrates an example in which current 425 and pulse width 426 are modulated in phase with a same modulation function and further illustrates a corresponding injected charge 427 for the waveform with the modulated current and modulated pulse width. The illustrated injected charge 427 provided by the in-phase modulation of both current 425 and pulse width 426 indicates that there is a compounding/exaggerated effect of stimulation at the peak of the oscillation and a greatly diminished effect of stimulation at the trough of the oscillation. For example, the illustrated current is modulated between 3 mA and 5 mA and the illustrated pulse width is modulated between 0.1 ms and 0.3 ms using a same modulation function. The in-phase modulated current and modulated pulse width may inject a charge ranging from about 0.3 μC (0.1 ms*3 mA) to about 1.5 μC (0.3 ms*5 mA).

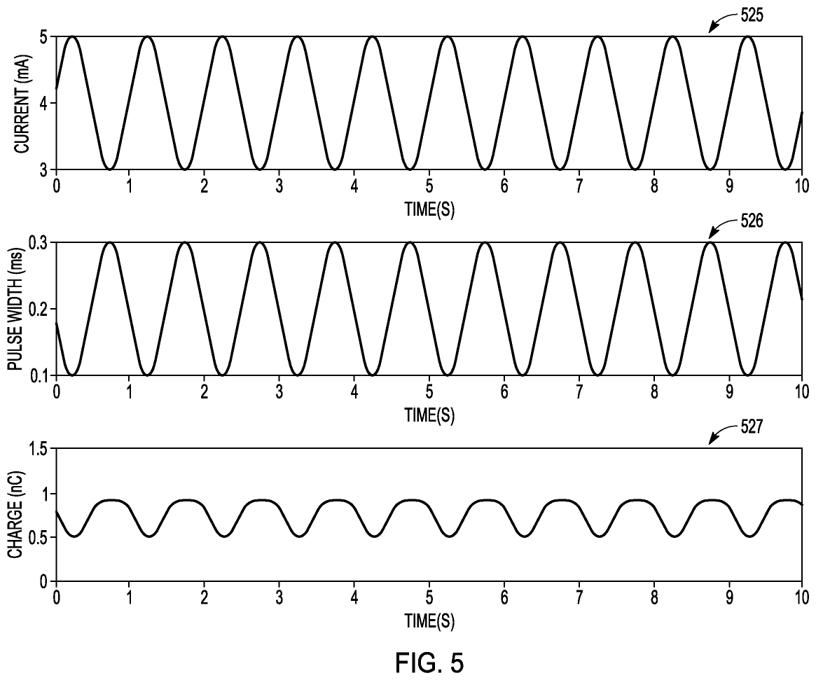

FIG. 5 illustrates an example in which current 525 and pulse width 526 are modulated out of phase with the same modulation function and further illustrates a corresponding injected charge 527 for the waveform with the out-of-phase modulated current and modulate pulse width. With reference to the injected charge 527, it can be seen that it is possible that the effects of stimulation can approach tonic stimulation and/or that resultant neural activity will exhibit a different periodicity than during in phase modulation. For example, the illustrated current is modulated between 3 mA and 5 mA and the illustrated pulse width is modulated between 0.1 ms and 0.3 ms using a same modulation function, but with the modulation 180 degrees out of phase. The out-of-phase modulated current and modulated pulse width may inject a charge ranging from about 0.5 μC (0.1 ms*5 mA) to about 0.9 μC (0.3 ms*3 mA).

The phase offset may allow the modulated parameter set to be tuned to increase or decrease the effect of the modulated parameters (e.g., amplitude and pulse width). Thus, instead of changing multiple dynamic stimulation parameters simultaneously to attenuate or increase the effects of dynamic stimulation, various embodiments of the present subject matter may adjust a single phase offset between two or more modulated parameters to achieve a similar result (e.g., as illustrated by way of examples in FIGS. 4 and 5, changing an injected charge from modulating between 0.3 μC and 1.5 μC to modulating between 0.5 μC and 0.9 μC by changing the phase between the modulated current and the modulated pulse width).

Various embodiments of the present subject matter may implement dynamic stimulation patterns modulated neurostimulation parameters and offset(s) between modulated neurostimulation parameter pairs. For example, various embodiments may implement dynamic stimulation patterns with multiple modulated parameters designed with phase offset(s) to minimize the change in current injection or amount of population activation. Instead of using the phase offset to minimize the charge injected or amount of population activation, some embodiments set the pulse width inversely with respect to amplitude to achieve a constant predetermined or user defined charge injection (in this case, 1 mC) for each stimulation. Other embodiments may implement dynamic stimulation patterns designed to provide a charge injection that periodically varies within a limited range (e.g., varies within a predefined minimum charge injection and a predefined maximum charge injection).

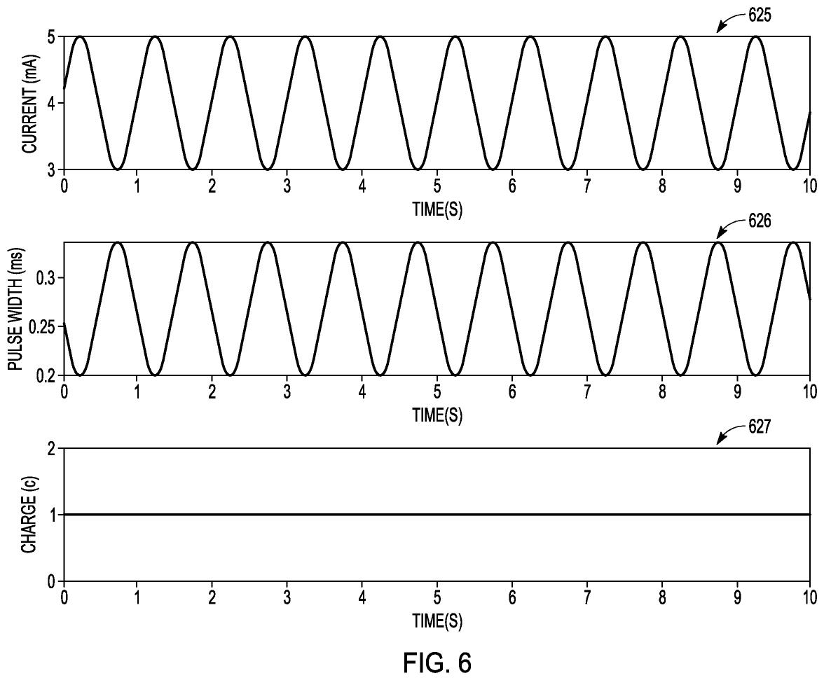

FIG. 6 illustrates an example in which modulated current 625 and modulated pulse width 626 are designed to provide a constant charge injection 627. The modulated current ranges between 3 mA and 5 mA and the pulse width is modulated between 0.2 ms and 0.33 or ⅓ ms to maintain a charge injection of about 1 nC (e.g., 0.2 ms*5 mA=1 μC and 3 ms*⅓ mA=1 μC. The modulating function for the pulse width is inversely related to amplitude.

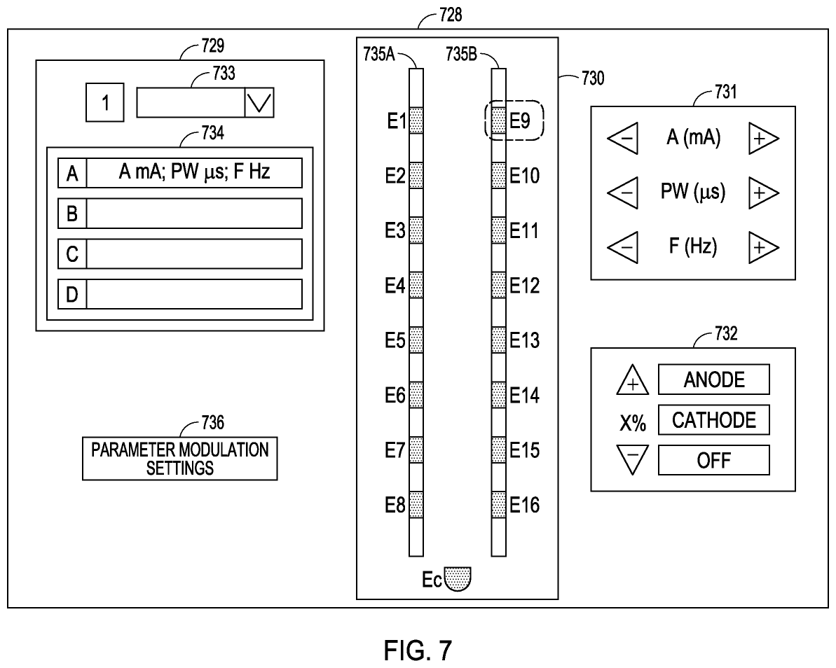

FIG. 7 illustrates, by way of example and not limitation, a programming screen display. The illustrated display 728 includes a program region 729, an electrode region 730, a pulse parameter programming region 731, and a fractionalization programming region 732. The program region 729 may include a stimulation program 733 selected from a set of one or more programs. The program selection is illustrated as a pull-down selection by way of example and not limitation. Other features for selecting a program may be implemented by the user interface. The neurostimulation system may have multiple timing channels, and the program region 729 may include user interface features for programming each of the timing channels (e.g., timing channels A-D) 734. Each timing channel 734 may be programmed with its own stimulation waveform. For example, each timing channel may be programmed with its own specific amplitude (mA), pulse width (ms) and frequency (Hz). The programming for each of the timing channels may be controlled using the pulse parameter programming region 731, which may be configured to program an amplitude, a pulse width and a frequency. The electrode region 730 may include an illustration of the electrodes. The illustration shows eight electrodes E1-E8 and E9-E16 on each of two leads 735A and 735B, as well as a “can” electrode EC on the housing of the implantable device. A user may select an electrode (e.g., E9), and use the fractionalization programming region 732 to program whether the selected electrode is an anode, a cathode, or off, and to also program the energy contribution of the electrode (e.g., as a percentage of the overall cathodic or anodic energy) for the selected timing channel in the program region 729.

As illustrated above, the stimulation parameters may be modulated by a modulation function. By way of example, a user interface feature (e.g., button) 736 may be selected to enter another screen display to program or modify at least some parameter modulation settings. However, it is contemplated that a screen display may be designed for programming or modifying at least some parameter modulation settings.

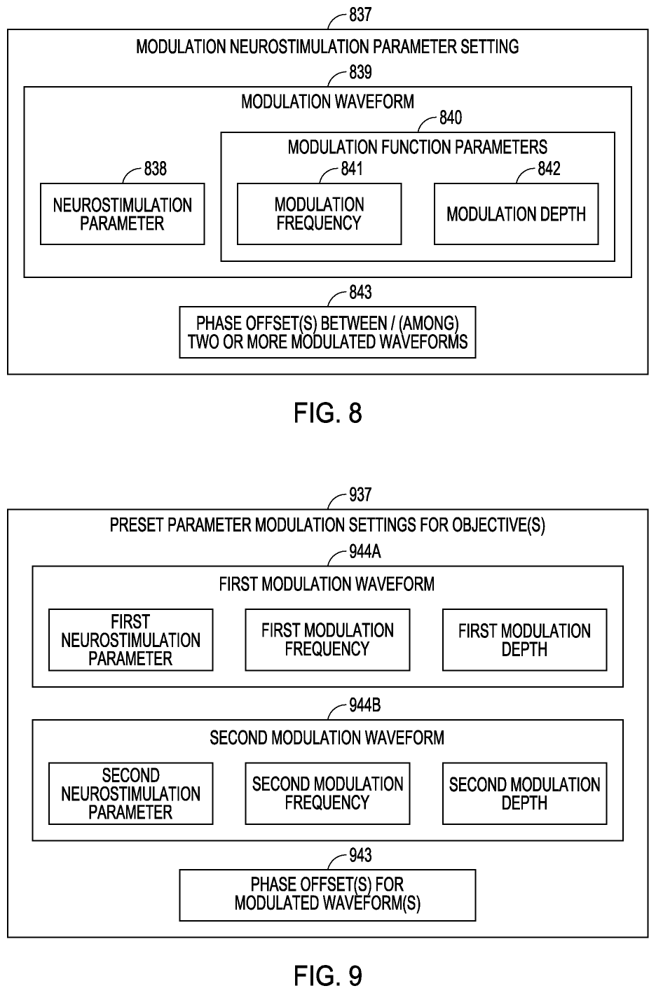

FIG. 8 illustrates, by way of example and not limitation, a modulated neurostimulation (or neuromodulation) setting 837. At least one neurostimulation parameter 838 may be modulated to provide a modulation waveform that includes a neurostimulator parameter 839 that is being modulated and further include modulation function parameters 840. For example, the modulation function parameters 840 may include a modulation frequency 841 and a modulation depth 842. Modulation frequency or rate indicates how quickly the parameter values repeat. For example, a sinusoidal or square waveform may provide a modulating envelope for the neurostimulation parameter. For example, in FIG. 4 the modulation of the amplitude repeats every “1” unit of time. The modulation depth indicates the amount that the value for the neurostimulation parameter varies during a modulation cycle. For example, in FIG. 4 the amplitude has an average value of 4 mA but varies +/−1 mA about the 4 mA. Phase offset(s) may be provided between or among modulated waveforms. The modulated neurostimulation parameter setting may include phase offset(s) 843.

Some embodiments provide the user with the ability to modify the modulation frequency and/or modulation depth. Some embodiments provide the user with the ability to select or change a previous selection of the neurostimulation parameter. The user interface may allow the user to access more than one modulated waveform. Some embodiments provide the user with the ability to select or otherwise determine phase offset(s) between or among two or more modulated waveforms.

FIG. 9 illustrates, by way of example and not limitation, preset parameter modulation settings designed to be effective in achieving neurostimulation objectives. The present parameter modulation settings 937 may include parameters for at least a first modulated waveform 944 and a second modulated waveform 945. The first modulated waveform 944 includes a neurostimulation parameter that is modulated. The neurostimulation parameter may include parameters for symmetric rectangular pulses or parameters for other asymmetric rectangular or other waveform shapes other than symmetric rectangular pulses. For example, the neuromodulation parameter may include at least one of an amplitude, a pulse width, a frequency, charge per pulse, a stimulation ON/OFF duty cycle, or electrode fractionalization such as may be used, by way of example and not limitation, for symmetric rectangular pulses. The neurostimulation parameter may include a waveform phase duration offset with amplitude adjustment, or variables for other waveform shape characteristics like slew rate, rate of change and the like such as may be used for shapes other than symmetric rectangular pulses. The second modulated waveform 945 includes another neurostimulation parameter that is modulated. Each of the first and second modulated waveforms may have a modulation frequency and a modulation depth for the neurostimulation parameter that is being modulated. The preset modulation parameters may include phase offset 943 between the first and second modulated waveforms. Some embodiments may have more than two modulated waveforms, and may have more than one phase offset to provide phase offsets between different modulated waveform pairs that provide a desired objective for the modulated waveforms. Preset modulation parameter settings may be provided, for example, if sequence modulation function parameters are already known or established beforehand. The preset settings may include preset angles (e.g., 180 degrees or T/2 offset, 90 degrees or T/4 offset, 45 degrees or T/8 offset, and the like). The settings may include an actual period of time, frequency, or proportions of the period T.

Some embodiments may allow a user to determine the modulation parameter settings using a modulation sweep. For example, the sweep may be performed in a clinical setting. The patient may experience a calibration ramp, where a phase offset is gradually applied to two or more parameters. The patient may mark most favorable patterns, and those offsets are kept. Sweeps may be performed for other modulation parameters, such as modulation frequency and/or modulation depth to allow the patient, clinician, device rep, or other user to determine when the modulated parameter settings achieve an objective for the therapy.

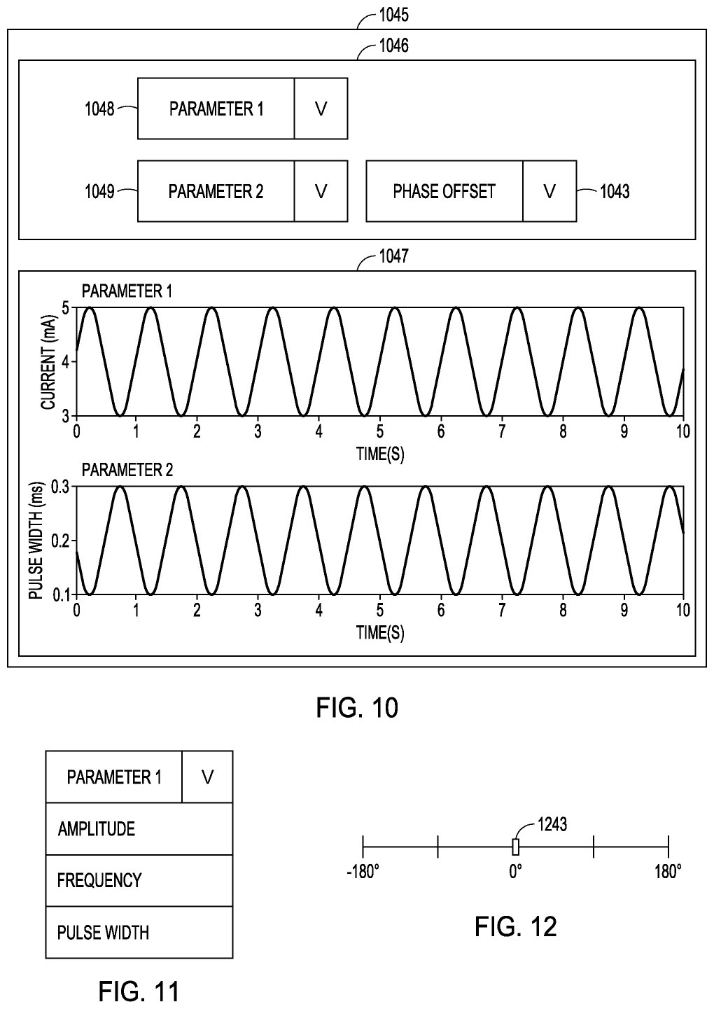

FIG. 10 illustrates, by way of example and not limitation, a screen display or a portion of screen display for programming or modifying parameter modulation settings. For example, selection of the button 736 in FIG. 7 may present the display similar to FIG. 10 on the user interface. Multiple parameters may be modulated by a modulation function to provide a modulation waveform. The illustrated display 1045 includes a modulation selection region 1046 and a modulation waveform display region 1047. In some embodiments, one or more of the modulated parameters are predetermined. In some embodiments, one or more of the modulated parameters 1048 and 1049 are user-selected. The illustrated display shows two user-selectable modulated parameters. The illustrated display also shows a user-selectable phase offset 1043 between the first and second parameters. The user selections are illustrated as using a pulldown menu. However, other user interface features may be used to provide these selections. The modulation waveform display may include a display of the first modulated waveform for the first parameter (e.g., current) and the second modulated waveform for the second parameter (e.g., pulse width). The modulation waveform display also may include a representation of the phase offset, such as by offsetting the second modulated waveform from the first modulated waveform.

FIG. 11 illustrates, by way of example and not limitation, a pulldown menu with parameters available to be selected for the first and/or second modulated parameters. In the illustrated example, the user is able to select among a modulated amplitude, a modulated frequency, or a modulated pulse width. Other neurostimulation parameter may be included in addition or alternative to these parameters. For example, the two or more modulated parameters may include two or more of an amplitude, a pulse width, a frequency, charge per pulse, a stimulation ON/OFF duty cycle, or electrode fractionalization.

FIG. 12 illustrates, by way of example and not limitation, a slider bar that may be used to select the phase offset. The slider bar 1243 may be used in place of the pulldown menu 1043 in FIG. 10, which may allow selection from two or more preset phase offsets. For example, user movement of the slider bar may result cause a movement of the second modulation waveform with respect to the first modulation waveform. Some embodiments may provide a real-time or near real-time movement of the second modulation waveform as the user moves the slider.

Other embodiments may be implemented to provide phase offset control. For example, a graphical user interface may include a slider bar, scroll bar, numeric display, and the like centered on zero, that used to define a phase offset between a modulated parameter pair. If modulation frequencies are the same for both parameters, a positive, non-zero modulation frequency and depth may be established. If modulation depth or modulation frequency are set to 0, some embodiments may disable the user's ability to a change phase offset. The offsetable parameters may include intensity settings: amplitude, pulse width, rate, charge delivered/pulse (combination of amplitude and pulse width). If charge/pulse is selected, then amplitude and pulse width may be excluded. One of the modulated parameters may be “anchored” at 0, serving as a reference while only the other is allowed to be “offset.” In another embodiment, the user can set the phase “initial condition” for each individual parameter modulation function manually. Minimum and maximum values shown can be based on half the period of the modulation function. The illustrated minimum and maximum values are −180° and +180°. Other graphical or numeric representations may be used to represent the phase offset, such as a representation of an “angle” of the offset or a time translation of the angle. Minimum and maximum labels may be −T/2 or +T/2, where T=1/modulation function frequency, etc. . . . .

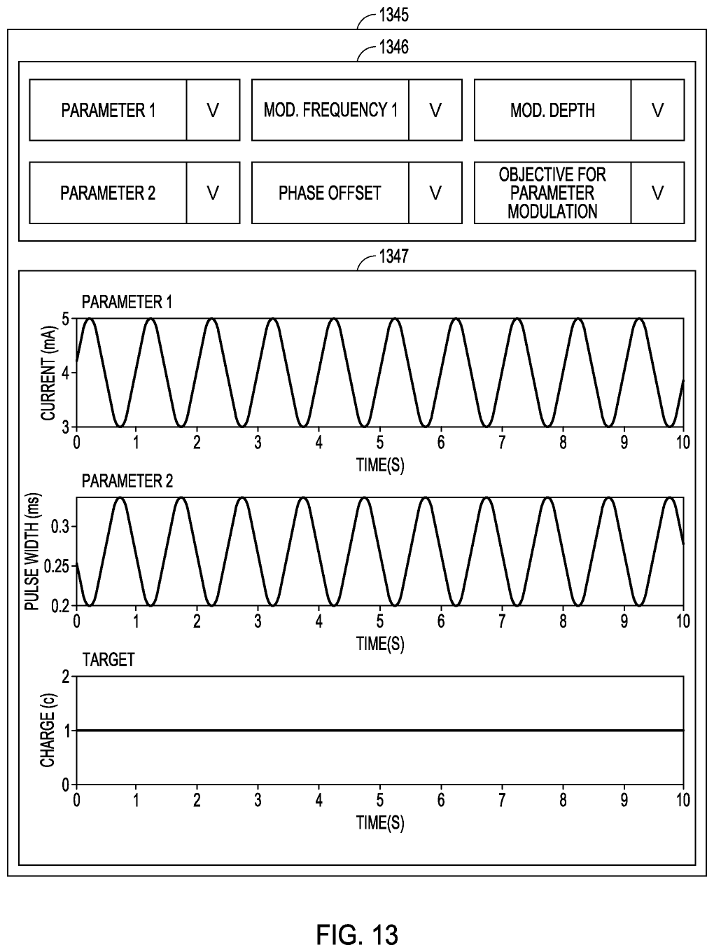

FIG. 13 illustrates, by way of example and not limitation, a screen display or a portion of screen display for programming or modifying parameter modulation settings. For example, selection of the button 736 in FIG. 7 may present the display similar to FIG. 13 on the user interface. The illustrated display 1346 includes a modulation selection region 1346 and a modulation waveform display region 1347. In some embodiments, one or more of the modulated parameters are predetermined. In some embodiments, one or more of the modulated parameters are user-selected. The illustrated display shows two user-selectable modulated parameters. The illustrated display also shows a user-selectable phase offset between the first and second parameters, a user-selectable modulation frequency, a user-selectable modulation depth and a user-selectable objective for the parameter modulation. Some embodiments may provide a preset modulation frequency, a preset modulation depth, a preset for the phase offset, and/or a preset for the objective. The illustrated modulation waveform display may include a representation of the first modulation waveform for the first parameter, a representation of the second modulation waveform for the second parameter with the phase offset, and a representation of the objective for the parameter modulation. For example, the objective may be to provide a constant or near constant charge injection. The representation of the objective may include a charge injection waveform. The illustrated representation of the charge waveform indicates that the first modulation waveform, the second modulation waveform, and the offset results in a constant or near constant charge injection. Representations for other objectives of the neurostimulation with modulated parameters may be illustrated. For example, an objective may include a population activation. Population activation provides an indicator of how “activated” the neurons are with different parameter. For example, an action potential may be induced, and rate determine a number of action potentials induced in the neurons over a given period of time. Other examples of objectives may be a rate of charge injection or a charge per pulse. Objectives may include a disease or symptoms of the disease to be treated. For example, a particular type of pain experienced in a particular part of the body may be effectively treated using a defined modulation parameter setting including a defined modulation of neuromodulation parameters and a defined offset between pairs of the modulated parameter waveforms. Similarly, other objectives may include a neural target to be stimulated or otherwise modulated, and physical effects to be achieved or avoided with the stimulation.

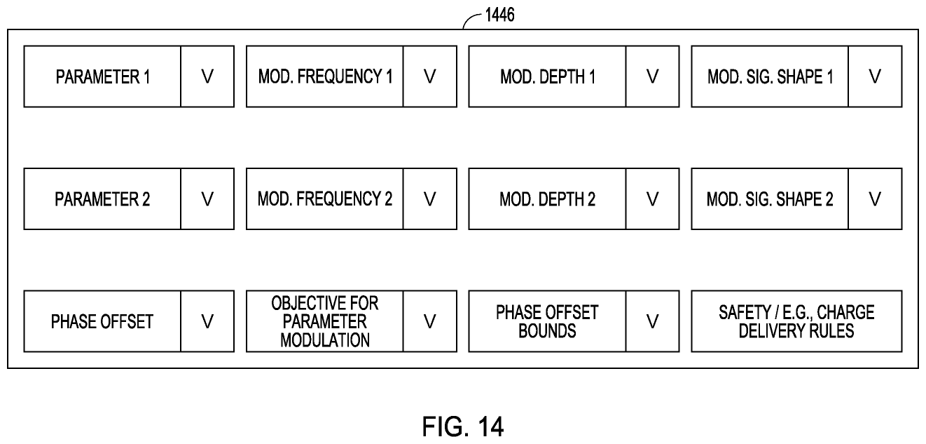

FIG. 14 illustrates by way of example and not limitation, a screen display or a portion of screen display for programming or modifying parameter modulation settings. For example, selection of the button 736 in FIG. 7 may present the display similar to FIG. 14 on the user interface. The illustrated display is an example of a modulation selection region 1446. The illustrated modulation selection region may include a user selection for at least two stimulations to be modulated, including the illustrated first neurostimulation parameter and the illustrated second neurostimulation parameter. Each of the user-selected parameters may include a user-selected modulation frequency for that parameter, a user-selected modulation depth for that parameter, and a user-selected modulation signal shape. A variety of modulation signal shapes may be used, including but not limited to sinusoidal waves, rectilinear pulse waves, sawtooth waves and the like. The modulation signal shape may be a shape corresponding to simple modulation function or a shape corresponding to a more complex modulation function. Thus, the user input may determine the waveform shape for an envelope function shape used to modulate the neurostimulation parameter(s) (e.g., parameter 1 or parameter 2 in FIG. 14). FIG. 14 also indicates that the screen display may include a user-selected phase offset and a user-selected objective for the parameter modulation. The screen display may also include a representation of phase offset bounds. Some embodiments provide the user with the ability to create and/or modify phase offset bounds. The screen display may also include a representation of safety rules such as charge delivery rules. Some embodiments provide the user with the ability to create and/or modify the safety rules.

The present subject matter may be used with stimulation parameters modulated using modulation function with a fixed period, although offset settings may need to be adjusted to account for the specific modulation function used. For example, an asymmetric sawtooth modulation would allow T/2 to +T/2 regardless, and a rectangular modulation function may allow either only fixed offset options (e.g., locked at “steps” in waveform) or continuous modulation over T/2 to T/2.

A user may be allowed to specify an arbitrary periodic modulation function over which modulation depth is controlled according to user defined bounds. Some embodiments may allow different frequencies to be used for different modulation functions. To accommodate, the system may account for different frequencies by displaying the phase offset control. The system may account for different frequencies by implementing modulation function phase offset bounds that limit a minimum offset between the modulation functions until they “wrap around” to each other again. For example, if a frequency modulation period is 3 seconds and an amplitude modulation period is 5 seconds, the minimum offset over the 15 second “wrap around” time may be the slower period T/5, where T is 5 seconds in this example. In some embodiments, the offset may be defined with respect to initial conditions rather than one or the other periods. For example, in waveforms with complex modulation schemes, the waveform itself and all elements/combinations within that waveform are “periodic” over either the least common multiple and/or the product of the individual periods. As such, an offset in terms of one or both of the periods may have the same effective impact as an overall time offset or an initial condition offset.

The safety rules may include safety interlocks that may need to be present to ensure charge delivery rules are not violated. A calculation may be run on a pulse-by-pulse basis to ensure that compliance voltage and charge delivery stay within approved safety limits, and a calculation may be performed at the very start of a modulation “period”, over all pulses, to ensure that direct current equivalent injection does not exceed approved limits. According to some embodiments, the output may be capped at the limit if a e.g., pulse train exceeds these limits. For example, the fastest and largest charge delivery periods of the waveform may be “throttled”, the stimulation may simply stop/exclude those portions of the waveform, and/or the device may throw a warning flag and prevent delivery of these settings.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
accessing a first modulated neuromodulation parameter waveform and a second modulated neuromodulation parameter waveform;
providing a phase offset between the first and the second modulated neurostimulation parameter waveforms to provide a modulated neurostimulation setting that includes the first and the second modulated neurostimulation waveforms and the phase offset; and
delivering neurostimulation according the modulated neurostimulation parameter setting.

2. The method of claim 1, further comprising receiving a user input corresponding to the phase offset, and determining the phase offset based on the user input.

3. The method of claim 2, wherein the user input includes an objective for the neurostimulation parameter setting, the method further comprising determining the phase offset from the objective.

4. The method of claim 1, further comprising:
accessing a neurostimulation setting having at least a first neurostimulation parameter and a second neurostimulation parameter; and
modulating the first parameter to provide the first modulated neurostimulation parameter waveform and modulating the second parameter to provide the second neurostimulation modulated neuromodulation parameter waveform.

5. The method of claim 4, wherein both the first neurostimulation parameter and the second neurostimulation parameter are modulated using a sinusoidal function.

6. The method of claim 5, further comprising receiving user input for at least one of a modulation frequency or a modulation depth, wherein at least one of the first modulated neurostimulation parameter waveform and the second modulated neurostimulation parameter waveform is at least partially defined using the received user input for the at least one of the modulation frequency or the modulation depth.

7. The method of claim 6, further comprising displaying in a single display screen on a user interface the first modulated neurostimulation parameter waveform, the second modulated neurostimulation parameter waveform, and the phase offset.

8. The method of claim 4, further comprising programming the neurostimulation setting, including programming a first value for the first neurostimulation parameter and programming a second value for the second neurostimulation parameter.

9. The method of claim 4, wherein the first and the second neurostimulation parameters include two or more of an amplitude, a pulse width, a frequency, charge per pulse, a stimulation ON/OFF duty cycle, or electrode fractionalization.

10. The method of claim 1, wherein the first and the second modulated neurostimulation parameter waveforms have a same modulation frequency.

11. The method of claim 1, wherein the first and the second modulated neurostimulation parameter waveforms have a different modulation frequency.

12. The method of claim 11, further comprising enforcing a minimum offset difference between the first and the second modulated neurostimulation parameter waveforms with the different modulation frequency.

13. The method of claim 1, further comprising receiving a user input to determine a waveform shape for a modulation function used to modulate at least one of the first neurostimulation parameter and the second neurostimulation parameter.

14. The method of claim 1, further comprising delivering the neurostimulation according to safety rules to prevent the modulated neurostimulation parameter setting from causing an unsafe dose of neurostimulation, and displaying a warning on a user interface if user input would cause the modulated neurostimulation parameter to be unsafe.

15. A non-transitory machine-readable medium including instructions, which when executed by a machine, cause the machine to perform a method comprising:
accessing a first modulated neurostimulation parameter waveform and a second modulated neurostimulation parameter waveform;
providing a phase offset between the first and the second modulated neurostimulation parameter waveforms to provide a modulated neurostimulation setting that includes the first and the second modulated neurostimulation parameter waveforms and the phase offset; and
delivering neurostimulation according the modulated neurostimulation parameter setting.

16. A system, comprising:
a neuromodulator and a programmer configured to program the neuromodulator to deliver neuromodulation according to a modulated neurostimulation parameter setting, wherein the modulated neurostimulation parameter setting includes a first modulated neurostimulation parameter waveform, a second modulated neurostimulation parameter waveform, and a phase offset between the first and the second modulated neurostimulation parameter waveforms.

17. The system of claim 16, wherein the programmer includes a user interface configured to receive a user input corresponding to the phase offset, wherein the programmer includes a controller operably connected to the user interface to determine the phase offset based on the user input.

18. The system of claim 16, wherein the programmer is configured to access a neurostimulation setting having at least a first neurostimulation parameter and a second neurostimulation parameter, modulate the first parameter to provide the first modulated neurostimulation parameter waveform, and modulate the second parameter to provide the second modulated neurostimulation parameter waveform.

19. The system of claim 16, wherein the processor is configured to receive a user input to determine a waveform shape for a modulation function used to modulate at least one of the first neurostimulation parameter and the second neurostimulation parameter.

20. The system of claim 16, wherein at least one of the programmer or the neurostimulator is configured to implement safety rules to prevent the modulated neurostimulation parameter setting from causing an unsafe dose of neurostimulation, and is configured to display a warning on a user interface if user input would cause the modulated neurostimulation parameter setting to be unsafe.
